(19) United States
(12) Patent Application Publication
(10) Pub. No.: US 2022/0240825 A1
(43) Pub. Date: Aug. 4, 2022

(54) Non-Invasive Urinary Output Measuring System

(71) Applicant: C. R. Bard, Inc., Franklin Lakes, NJ (US)

(72) Inventors: Reagan H. Belflower, Atlanta, GA (US); Matthew B. Blagburn, Roswell, GA (US); Dana A. Abdelal, Marietta, GA (US)

(21) Appl. No.: 17/587,938

(22) Filed: Jan. 28, 2022

Related U.S. Application Data
(60) Provisional application No. 63/144,381, filed on Feb. 1, 2021.

(51) Int. Cl. A61B 5/20; A61B 5/00

(52) U.S. Cl. CPC A61B 5/208; A61B 5/0059; A61B 5/742

(57) ABSTRACT

Disclosed herein is a non-invasive urinary output measuring system having a urinary output measuring device and a fluid sensor system. The urinary output measuring device includes a receptacle body having a proximal opening, the receptacle body defining a collection cavity configured to receive a volume of fluid. The collection cavity includes a distal opening and an internal hydrophobic surface. The fluid sensor system is configured to measure the volume of fluid and includes a fluid sensor, a console including a processor further includes non-transitory computer-readable medium having a plurality of logic modules, one or more light indicator, and an opening mechanism configured to detachably seal the distal opening, the opening mechanism including a cover coupled to the receptacle body, and a lever configured to transition the opening mechanism between a closed configuration and an opened configuration.

### PRIORITY

This application claims the benefit of priority to U.S. Provisional Application No. 63/144,381, filed Feb. 1, 2021, which is incorporated by reference in its entirety into this application.

### BACKGROUND

Non-invasively measuring urinary output for ambulatory patients can be difficult. Most methods use urine hats, which require patients to be in close proximity to a bathroom during use. In using urine hats, some of the collected fluid may not funneled to the bottom of the urine hat, leading to inconsistent measurements. Furthermore, disposing of the urine collected within the urine hat can be messy. It would be beneficial to the patient and the clinician to have a non-invasive urinary output measuring system that can obtain accurate measurements and easily dispose of collected urine. Disclosed herein is a system and method of use that address the foregoing.

### SUMMARY

Disclosed herein is a non-invasive urinary output measuring system having a urinary output measuring device and a fluid sensor system. The urinary output measuring device includes a receptacle body having a proximal opening, the receptacle body defining a collection cavity configured to receive a volume of fluid. The collection cavity includes a distal opening and an internal hydrophobic surface. The fluid sensor system is configured to measure the volume of fluid and includes a fluid sensor, a console including a processor further includes non-transitory computer-readable medium having a plurality of logic modules, one or more light indicator, and an opening mechanism configured to detachably seal the distal opening, the opening mechanism including a cover coupled to the receptacle body, and a lever configured to transition the opening mechanism between a closed configuration and an opened configuration.

In some embodiments, the receptacle body includes a collection port having a lumen, the collection port extending from the collection cavity to an external surface of the receptacle body.

In some embodiments, the receptacle body includes a seat, and in some embodiments the lever is coupled to the seat.

In some embodiments, the opened configuration includes the lever in a distal position and the cover is detached from the distal opening.

In some embodiments, the closed configuration includes the lever in a proximal position and the cover seals the distal opening.

In some embodiments, the fluid sensor includes a laser fluid sensor or an ultrasound fluid sensor.

In some embodiments, the fluid sensor is coupled to the seat or to the internal hydrophobic surface.

In some embodiments, the one or more light indicators are coupled to the seat.

In some embodiments, the console is coupled to the seat or the receptacle body.

In some embodiments, the opening mechanism includes an activation lock configured to secure the opening mechanism in the closed configuration.

In some embodiments, the plurality of logic modules, when executed by the processor, are configured to perform operations including detecting a volume of fluid within the collection cavity, measuring a volume of fluid within the collection cavity, associating a volume measurement value with a time of day value in a volume measurement value-time of day value pairing, illuminating the light indicator a first color, activating an activation lock, illuminating the light indicator a second color, and transmitting the volume measurement value-time of day value pairing to a display or a computing device.

In some embodiments, the light indicator is configured to illuminate the first color when the console is obtaining a volume measurement value.

In some embodiments, the activation lock is activated while the light indicator illuminates the first color.

In some embodiments, the light indicator is configured to illuminate the second color when the console has received the volume measurement value-time of day value pairing or the console has transmitted the volume measurement value-time of day value pairing to the display or the computing device.

In some embodiments, the internal hydrophobic surface includes a plurality of super hydrophobic structures.

In some embodiments, the opening mechanism is biased to the closed configuration.

In some embodiments, the cover includes a hinge.

Also disclosed herein is a method of measuring a volume of fluid from a user including acquiring a volume of fluid within a collection cavity of a non-invasive urinary output monitoring system, the collection cavity having an internal hydrophobic surface, illuminating a light indicator of a fluid sensor system, a first color, detecting a volume measurement value using the fluid sensor system, illuminating the light indicator a second color, and disposing of the volume of fluid using an opening mechanism.

In some embodiments, illuminating a light indicator of the fluid sensor system, a first color includes illuminating the light indicator in communication with a console a first color.

In some embodiments, illuminating a light indicator of the fluid sensor system, a first color includes illuminating the light indicator coupled to a seat of a urinary output measuring device of the non-invasive output monitoring system, a first color.

In some embodiments, detecting the volume measurement value using the fluid sensor system includes detecting the volume measurement value using a fluid sensor in communication with the console.

In some embodiments, detecting the volume measurement value using the fluid sensor system includes detecting the volume measurement value using the fluid sensor wherein the fluid sensor is a laser fluid sensor or an ultrasound fluid sensor.

In some embodiments, detecting the volume measurement value using the fluid sensor system includes detecting using the fluid sensor when the fluid sensor is coupled to a seat or to the internal hydrophobic surface.

In some embodiments, disposing of the volume of fluid using an opening mechanism includes obtaining a sample of the volume of fluid through a lumen of a collection port extending from the collection cavity to an external surface of a receptacle body having the collection cavity therein.

In some embodiments, disposing of the volume of fluid using the opening mechanism includes transitioning the opening mechanism from a closed configuration to an opened configuration to dispose of the volume of fluid.

In some embodiments, transitioning the opening mechanism from the closed configuration to the opened configuration includes moving a lever from a proximal position in the closed configuration to a distal position in the opened configuration.

In some embodiments, transitioning the opening mechanism from the closed configuration to the opened configuration includes a cover configured to detachably seal a distal opening of the collection cavity transitioning from a closed configuration sealing the volume of fluid in the collection cavity to an opened configuration allowing the volume of fluid to exit the collection cavity.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a non-invasive urinary output measuring system, in accordance with some embodiments.

FIG. 2 illustrates a cross sectional view of the urinary output measuring device, the fluid sensor system and the opening mechanism, in accordance with some embodiments.

FIG. 3 illustrates a block diagram of some components of the fluid sensor system, in accordance with some embodiments.

FIGS. 4A-4D illustrate an exemplary method of collecting, measuring and disposing of a volume of fluid using the non-invasive urinary output measuring system, in accordance with some embodiments.

FIG. 5 illustrates a flow chart of the exemplary method of measuring a volume of fluid from a user, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

Unless defined otherwise, all technical and scientific terms used herein have the same meaning as commonly understood by those of ordinary skill in the art.

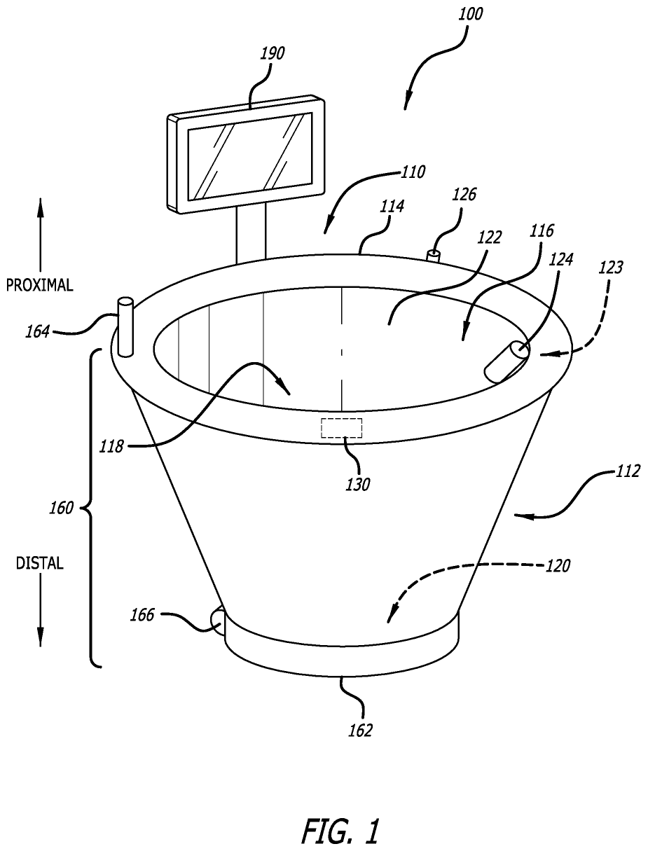

FIG. 1 illustrates a perspective view of a non-invasive urinary output measuring system 100, in accordance with some embodiments. In some embodiments, the non-invasive urinary output measuring system (“system”) 100 includes a urinary output measuring device (“device”) 110 having a receptacle body 112. In some embodiments, the system 100 including the device 110 may be configured to collect, accurately and quickly measure, and dispose of a volume of fluid, including body fluids, from a patient. Exemplary body fluids can include urine, blood, interstitial fluid, peritoneal fluid, saliva, mucus, or the like. As used herein, a volume of fluid includes a volume of urine voided from an user. In some embodiments, the receptacle body 112 includes a seat 114 thereon. In some embodiments, the seat 114 may be located over a proximal opening 116. The seat 114 may be used by the user while the user is voiding a volume of urine therein. The receptacle body 112 defines a collection cavity 118 therein. The collection cavity 118 may be configured to receive and hold a volume of urine to be measured. The collection cavity 118 may have a distal opening 120 configured to allow the volume of urine to pass therethrough. In some embodiments, the collection cavity 118 may include an internal hydrophobic surface 122 wherein a portion of the internal hydrophobic surface 122 or the entire internal hydrophobic surface 122 may be configured to include non-stick or hydrophobic properties thereon that will be described in more detail herein.

The system 100 includes a fluid sensor system 123 configured to measure the volume of urine within the collection cavity 118. In some embodiments, the fluid sensor system 123 includes a fluid sensor 124 in communication with a console 130 and one or more light indicators 126. In some embodiments, the fluid sensor 124 may be coupled to the seat 114 or coupled to the internal surface 122 of the collection cavity 118. The one or more light indicators 126 may be configured to indicate to a user the status of the fluid sensor 124 as will be described in more detail herein. In some embodiments, the one or more light indicators 126 may be coupled to the seat 114 or may be coupled to the receptacle body 112 and may be visible to a user while the user is voiding a volume of fluid. In some embodiments, the console 130 may be coupled to the seat 114, coupled to the receptacle body 112 or coupled to a display 190. In some embodiments, the system 100 includes the display 190 and the display may be coupled to the receptacle body 112 or the seat 114. In some embodiments, the display 190 may be detachably coupled to the receptacle body 112 or the seat 114. In some embodiments, the display 190 may be wired to the console 130 or may be in wireless communication with the console 130. In some embodiments, the display 190 may be configured to display a volume measurement value of the volume of urine within the collection cavity 118.

The system 100 further includes an opening mechanism 160 coupled to the receptacle body 112. In some embodiments, the opening mechanism 160 includes a cover 162 configured to detachably seal the distal opening 120 and a lever 164 configured to transition the opening mechanism 160 between a closed configuration and an opened configuration. In some embodiments, the cover 162 may include a hinge 166 configured to couple the cover 162 to the receptacle body 112. In some embodiments, the lever 164 may be coupled to the seat 114 or the receptacle body 112. The opening mechanism 160 may be configured to be in a closed configuration, wherein the cover 162 seals the distal opening 120, maintaining the volume of urine within the collection cavity 118 and the lever 164 is in a first proximal position. The opening mechanism 160 may be configured to be in an opened configuration, wherein the seal between the cover 162 and the distal opening 120 is broken, allowing the volume of urine to exit the collection cavity 118 through the distal opening 120 to be disposed of, and the lever 164 is in a second distal position.

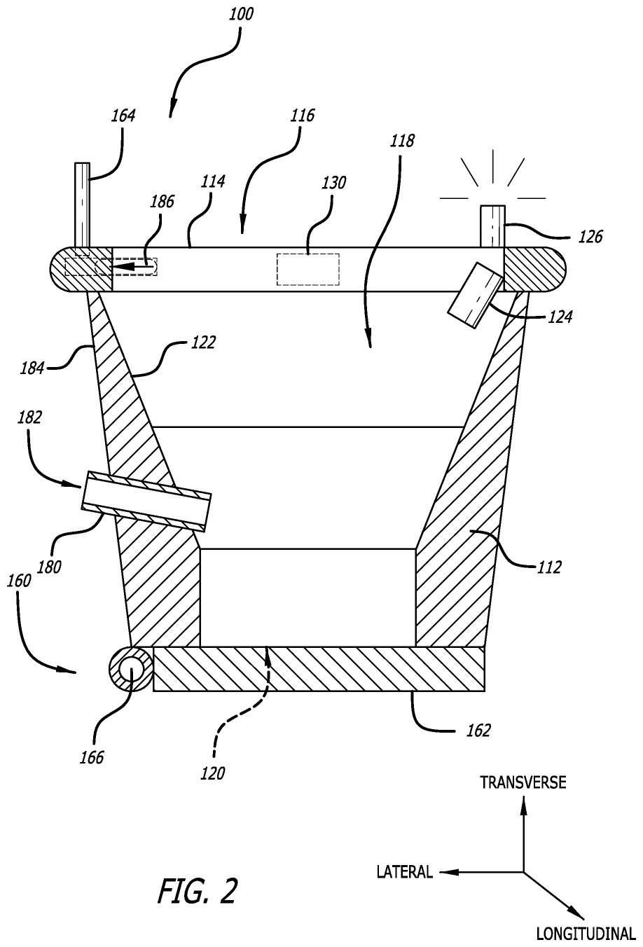

FIG. 2 illustrates a cross sectional view of the urinary output measuring device 110, the fluid sensor system 123 and the opening mechanism 160, in accordance with some embodiments. In some embodiments, the device 110 includes the seat 114, the proximal opening 116, the collection cavity 118 having the distal opening 120. In some embodiments, the receptacle body 112 may be in the shape of a rectangular prism, a cylinder, a triangular prism, an inverted cone or the like. In some embodiments, the collection cavity 118 may be in the shape of an inverted cone, an inverted hemisphere, an inverted triangular prism, a cylinder or the like. In some embodiments, the shape of the collection cavity 118 may be configured to allow the volume of urine to move through the collection cavity 118 to the distal opening 120 by way of gravity flow. In some embodiments, the shape of the receptacle body 112 and the shape of the collection cavity 118 may be the same or the shape of the receptacle body 112 and the shape of the collection cavity 118 may be different. The system 100 includes the fluid sensor system 123 including the fluid sensor 124 coupled to the collection cavity 118, the console 130 in communication with each of the fluid sensor 124 and the one or more light indicators 126, coupled to the seat 114.

The device 110 further includes the opening mechanism 160 including the cover 162 coupled to the lever 164. In some embodiments, the lever 164 may be used to transition the opening mechanism 160 between the closed configuration and the opened configuration. In some embodiments, the lever 164 may be configured to be depressed distally, moving from the first proximal position to the second distal position, to transition the cover 162 between the closed configuration and the opened configuration. In some embodiments, the cover 162 may be configured to biased towards the closed configuration. In some embodiments, the lever 164 may include an activation lock 186 configured to prevent distal movement of the lever 164 until the fluid sensor 124 is no longer activated. In some embodiments, the activation lock 186 may be configured to move laterally outward to prevent distal movement of the lever 164 or move laterally inward to allow distal movement of the lever 164. In some embodiments, once the opening mechanism 160 has transitioned from the opened configuration to the closed configuration after the volume of urine has been disposed of, the activation lock 186 may reset to secure the lever 164 in the first proximal position.

In some embodiments, the device 110 includes a collection port 180 in fluid communication with the collection cavity 118. In some embodiments, the collection port 180 may be a cylindrical port, having a lumen 182 therethrough, the collection port 180 extending from the collection cavity 118 to an external surface 184 of the receptacle body 112. In some embodiments, the collection port 180 may be used to acquire a sample of the volume of urine collected in the collection cavity 118 after the volume of urine has been measured. In some embodiments, the collection port 180 may extend laterally to the external surface 184 or may be angled towards the seat 114 to facilitate an easier acquisition of a sample of the volume of urine within in the collection cavity 118 and to ensure no portion of the volume of urine is lost through the collection port 180.

In some embodiments, the fluid sensor 124 may be coupled to the seat 114 or coupled to the internal hydrophobic surface 122 of the collection cavity 118. In some embodiments, the fluid sensor 124 may be angled towards the distal opening 120 within the collection cavity 118. In some embodiments, the fluid sensor 124 includes a laser fluid sensor or an ultrasound fluid sensor. In some embodiments, the one or more light indicators 126 may include one or more light emitting diodes (“LEDs”). In some embodiments, the light indicators 126 may include bi-color LEDs.

In some embodiments, the internal hydrophobic surface 122 of the collection cavity 118 includes non-stick or hydrophobic properties thereon, configured to direct the entirety of the volume of urine to the distal portion of the collection cavity 118. In some embodiments, the hydrophobic properties may include compounds, substances or solutions such as perfluoroalkyl and polyfluoroalkyl substances, perfluoropolyether, perfluoropolyether-tetrafluorethylene, ceramics, silica, titania, other polymers or the like. In some embodiments, the internal hydrophobic surface 122 may include a plurality of super hydrophobic structures thereon configured to direct the entirety of the volume of urine to the distal portion of the collection cavity 118. In some embodiments, the hydrophobic properties such as the compounds, substances or solutions may be sprayed onto, dripped onto, dipped into, painted onto the internal hydrophobic surface 122 of the collection cavity 118. Other mechanisms of generating the hydrophobic properties onto the internal hydrophobic surface 122 are also considered. In some embodiments, a portion of, or the entirety of the collection cavity 118 may be constructed of same materials as the internal hydrophobic surface 122. In some embodiments, the combination of the shape of the collection cavity 118 and the internal hydrophobic surface 122 having the non-stick or hydrophobic properties thereon may be configured to direct the entirety of the volume of urine to the distal portion of the collection cavity 118 for an accurate measurement of the volume of urine collected therein. For example, a hydrophobic coating could be disposed on the surface, or a sealer could be disposed similar to Rain-X®.

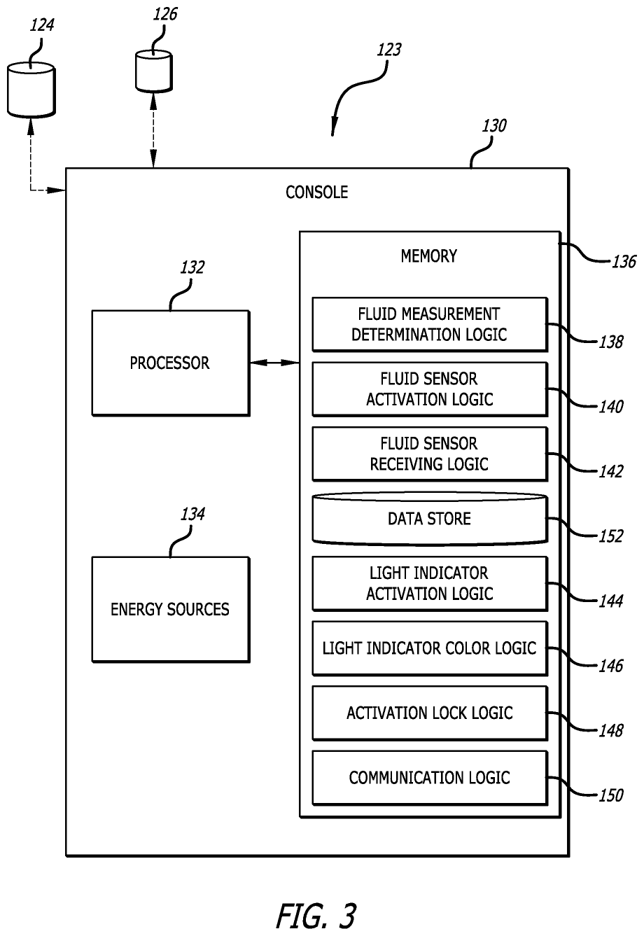

FIG. 3 illustrates a block diagram of some components of the fluid sensor system 123, in accordance with some embodiments. The fluid sensor system 123 includes the fluid sensor 124 and the one or more light indicators 126, each in communication with the console 130. In some embodiments, the console 130 may be wired to the fluid sensor 124 and may be wired to the one or more light indicators 126. In some embodiments, the console 130 may be in wireless communication with the fluid sensor 124 and the one or more light indicators 126. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. In some embodiments, the console 130 may be coupled to the external surface 184 of the receptacle body 112, may be coupled to the seat 114, may be coupled to the internal surface 122 or may be coupled to the display 190. In some embodiments, the console 130 may include one or more processors 132, one or more energy sources 134, and non-transitory, computer-readable medium (“memory”) 136 including a plurality of logic modules.

In some embodiments, the plurality of logic modules may include one or more of: a fluid measurement determination logic 138, a fluid sensor activation logic 140, a fluid sensor receiving logic 142, a light indicator activation logic 144, a light indicator color logic 146, an activation lock logic 148, a communications logic 150, and a data store 152. In some embodiments, the plurality of logic modules, when executed by the processor 132, may be configured to perform operations including: detecting a volume of fluid within the collection cavity 118, measuring the volume of fluid within the collection cavity 118, associating a volume measure value with a time of day value in a volume measurement value-time of day value pairing, illuminating the one or more light indicators 126 a first color, activating the activation lock 186, illuminating the one or more light indicators 126 a second color, and transmitting the volume measurement value-time of day value pairing to the display 190 or a computing device.

In some embodiments, the fluid measurement determination logic 138 may be configured to determine if the entirety of the volume of urine is within the collection cavity 118 by using the fluid sensor 124 to take multiple volume measurements in a short time period. In some embodiments, the fluid measurement determination logic 138 may be configured to detect fluid movement in 5 second intervals, although greater or less increments are also contemplated. In some embodiments, if multiple consecutive measurements are the same value or within an established threshold, it will be determined that the entirety of the volume of urine has been collected within the collection cavity 118 and the fluid measurement determination logic 138 may activate the fluid sensor activation logic 140. In some embodiments, the fluid sensor activation logic 140 may be configured to activate the fluid sensor 124, to measure the volume of urine within the collection cavity 118 and generate a volume measurement value. In some embodiments, the fluid sensor activation logic 140 may transmit the volume measurement value to the console 130. In some embodiments, the fluid sensor receiving logic 142 may be configured to receive the volume measurement value and associate the volume measurement value with a time of day value in a {volume measurement value, time of day value} pairing. The time of day value is the time of day the volume measurement value was generated. In some embodiments, the light indicator activation logic 144 may be configured to illuminate a first color in the one or more light indicator 126.

In some embodiments, the fluid sensor activation logic 140 may also activate the light indicator activation logic 144. In some embodiments, the light indicator color logic 146 may be configured illuminate a second color in the one or more light indicators 126, indicating a volume measurement value has been acquired and that the user may dispose of the volume of urine within the collection cavity 118. In some embodiments, the light indicator color logic 146 may be configured to change the light indicators from the second color to the first color. In some embodiments, the first color and the second color may be user specified or automatically generated by the console 130. In some embodiments, the activation lock logic 148 may be configured to activate the activation lock 186 to lock the lever 164 in a proximal position until the light indicator 126 changes from the first color to the second color, to prevent a user from pre-emptively transitioning the opening mechanism 160 from the closed configuration to the opened configuration and disposing of the volume of urine before the volume measurement value is obtained. In some embodiments, the communications logic 150 may be configured to transmit the volume measurement value-time of day value pairing to the display 190, a computing device, or an electronic medical record system. In some embodiments, the data store 152 may be configured to store the {volume measurement value, time of day value} pairings. In some embodiments, the fluid sensor system 123 may be configured to measure the volume of urine within the device 110 at regular time intervals or user defined time intervals (e.g., every 15 minutes).

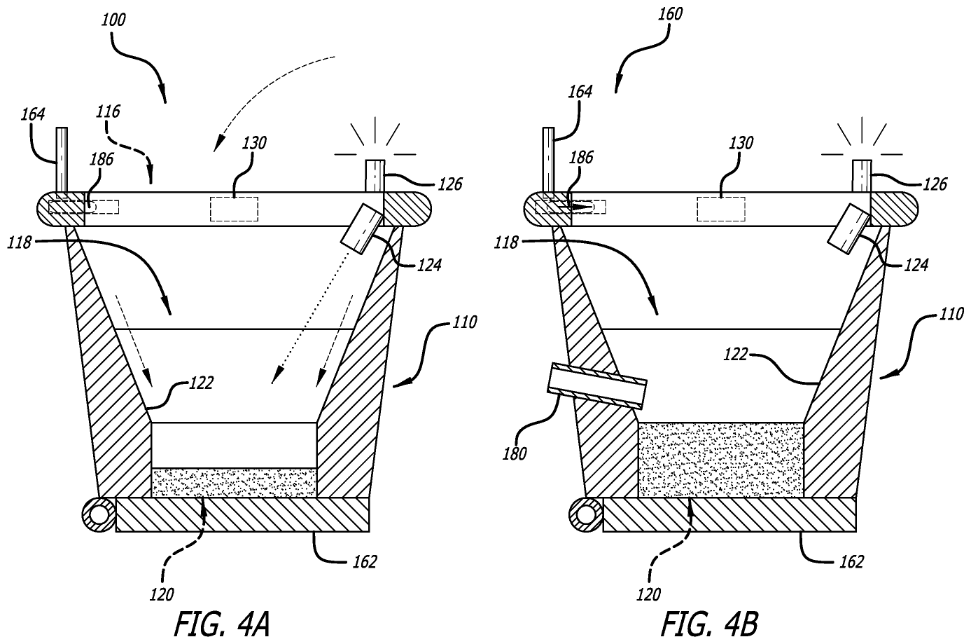

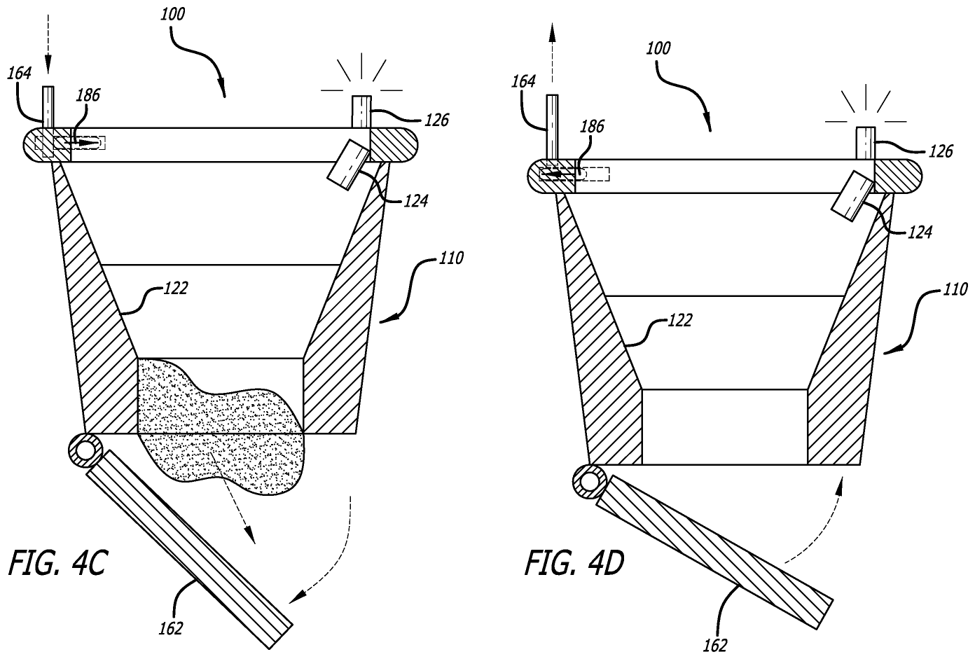

FIGS. 4A-4D illustrate an exemplary method of collecting, measuring and disposing of a volume of fluid, in accordance with some embodiments. In some embodiments, as illustrated in FIG. 4A, the user may void a volume of urine into the collection cavity 118 through the proximal opening 116 of the non-invasive urinary output monitoring device 110. In some embodiments, a portion of the volume of urine may encounter the internal hydrophobic surface 122 having the non-stick or hydrophobic properties thereon and slide down the collection cavity 118 to the distal portion of the collection cavity 118. Once the entirety of the volume of urine is acquired in the collection cavity 118, the one or more light indicators 126 may be configured to illuminate a first color and the fluid sensor 124 may be configured to acquire the volume measurement value. In some embodiments, the fluid sensor 124 may generate a laser beam towards the surface of the volume of urine collected at the distal portion of the collection cavity 118. The laser beam may be reflected from the surface of the volume of urine back to the fluid sensor 124. The time between the laser beam being generated by the fluid sensor 124 and the reflection of the laser beam being detected by the fluid sensor 124 may be configured to correlate to a volume of urine within the collection cavity 118, and indicated as the measurement volume value. In some embodiments, the fluid sensor 124 may transmit the volume measurement value to the console 130. In some embodiments, the activation lock 186 may be activated to secure the lever 164 in the proximal position while the fluid sensor 124 is acquiring the volume measurement value.

As illustrated in FIG. 4B, once the entirety of the volume of urine is collected into the collection cavity 118 and the fluid sensor 124 acquires the volume measurement value, the one or more light indicators 126 may be configured to change from the first color to the second color, indicating the volume of urine may be disposed of. In some embodiments, the user or a clinician may be configured to collect a sample of the urine through the collection port 180 before the user disposes of the volume of urine. In some embodiments, once the one or more light indicators 126 change to the second color, the activation lock 186 may be disabled by moving laterally inward, to allow distal movement of the lever 164. Once the volume measurement value is acquired and the one or more light indicators 126 changes to the second color, the user may transition the opening mechanism 160 from the closed configuration to the opened configuration. As illustrated in FIG. 4C, the lever 164 may be depressed distally to transition the cover 162 from the closed configuration to the opened configuration by moving the cover 162 away from the distal opening 120, allowing the volume of urine to move out of the distal opening 120. In some embodiments, the volume of urine may be disposed of into a sanitary hardware device (e.g., a toilet). As illustrated in FIG. 4D, once the entirety of the volume of urine has been disposed of through the distal opening 120, the cover 162 may be configured to return to the closed configuration. In some embodiments, as the cover 162 is returned to the closed configuration, the lever 164 may be reset, in a proximal position. In some embodiments, once the cover 162 is returned to the closed configuration, the light indicator 126 may be configured to return to the first color or may cease to be illuminated. In some embodiments, once the cover 162 is returned to the closed configuration, the activation lock 186 may be activated. Advantageously, the shape of the collection cavity 118 and the internal hydrophobic surface 122 having the non-stick or hydrophobic properties thereon allow the entirety of the volume of urine to be funneled toward the distal portion of the collection cavity 118 for accurate measurement and alleviates the necessity of a volume of fluid to flush the collection cavity 118 between uses.

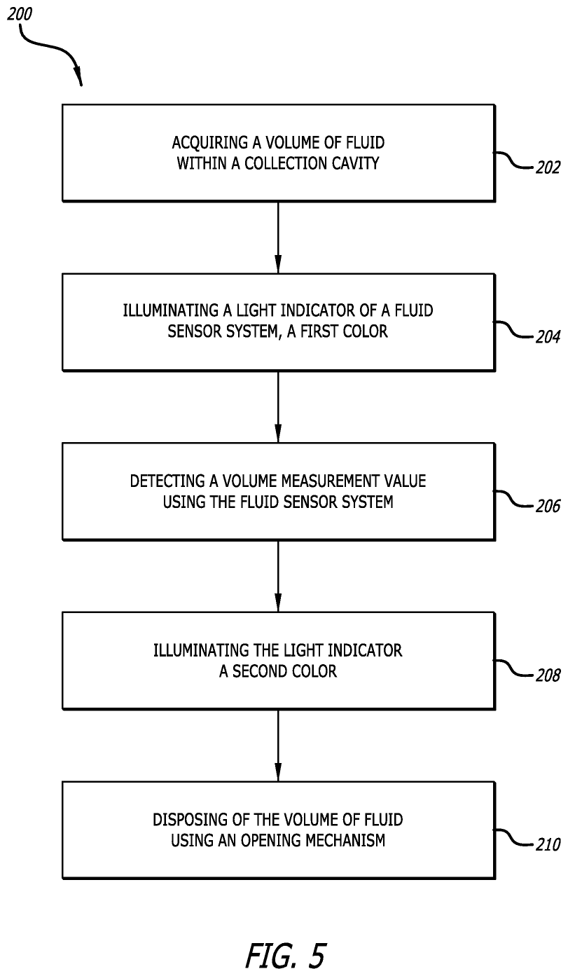

FIG. 5 illustrates a flow chart of the exemplary method of measuring a volume of fluid from a user, in accordance with some embodiments. In some embodiments, the method 200 includes acquiring a volume of fluid with a non-invasive urinary output monitoring system 100 (block 202). In some embodiments, acquiring includes a user voiding a volume of fluid (e.g., urine) into the collection cavity 118 of the urinary output monitoring device 110 of the system 100. In some embodiments, the collection cavity 118 may include the internal hydrophobic surface 122 having non-stick or hydrophobic properties thereon wherein the collection cavity 118 is shaped in an inverted cone and configured to funnel the entirety of the volume of urine towards a distal portion of the collection cavity 118. In some embodiments, the method 200 includes illuminating a light indicator 126 of a fluid sensor system 123, a first color (block 204). In some embodiments, illuminating the light indicator 126 a first color indicates to a user that the volume of urine within the collection cavity 118 has not yet been measured and to not yet dispose of the volume of urine. In some embodiments, illuminating the light indicator 126 of the fluid sensor system 123 a first color includes the light indicator 126 being coupled to the seat 114 and being in communication with the console 130.

In some embodiments, the method 200 include detecting a volume measurement value using the fluid sensor system 123 (block 206). In some embodiments, the fluid sensor system 123 includes the console 130 in communication with each of the laser fluid sensor 124 and the light indicator 126. In some embodiments, detecting the volume measurement value using the fluid sensor system 123 includes using the fluid sensor 124 being coupled to the seat 114 or the internal hydrophobic surface 122. In some embodiments, detecting the volume measurement value using the fluid sensor system 123 includes using the laser fluid sensor 124 to generate a laser directed towards the surface of the volume of urine and detect the reflected laser from the surface of the volume of urine. In some embodiments, detecting a volume measurement value includes associating the volume measurement value with a time of day value and generating a {volume measurement value, time of day value} pairing. In some embodiments, detecting includes transmitting the {volume measurement value, time of day value} pairing to the console 130. In some embodiments, detecting includes transmitting the {volume measurement value, time of day value} pairing to the display 190, a computing device or the like.

In some embodiments, the method 200 includes illuminating the light indicator 126 a second color (block 208). In some embodiments, illuminating the light indicator a second color may be configured to indicate to the user that the volume measurement value has been detected and the volume of urine may be disposed of.

In some embodiments, the method 200 includes disposing of the volume of fluid using the opening mechanism 160 (block 210). In some embodiments, disposing of the volume of fluid includes obtaining a sample of the volume of fluid through the lumen 182 of the collection port 180 extending from the collection cavity 118 to the external surface 184 of the receptacle body 112.

In some embodiments, disposing of the volume of fluid using the opening mechanism 160 includes transitioning the opening mechanism 160 from the closed configuration to the opened configuration to dispose of the volume of fluid. In some embodiments, transitioning the opening mechanism 160 from the closed configuration to the opened configuration includes moving the lever 164 from a proximal position in the closed configuration to a distal position in the opened configuration. In some embodiments, transitioning the opening mechanism 160 from the closed configuration to the opened configuration includes the cover 162 configured to detachably seal the distal opening 120 of the collection cavity 118 transitioning from the closed configuration sealing the volume of fluid in the collection cavity 118 to the opened configuration allowing the volume of fluid to exit the collection cavity 118. In some embodiments, disposing of the volume of fluid using the opening mechanism 160 includes disposed of the volume of fluid in a sanitary hardware device (e.g., a toilet).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A non-invasive urinary output measuring system, comprising:
a urinary output measuring device, comprising:
a receptacle body having a proximal opening, the receptacle body defining a collection cavity configured to receive a volume of fluid, the collection cavity having a distal opening and an internal hydrophobic surface;
a fluid sensor system configured to measure the volume of fluid, the fluid sensor system comprising:
a fluid sensor;
a console including a processor;
non-transitory computer-readable medium having stored thereon a plurality of logic modules; and
one or more light indicators; and
an opening mechanism configured to detachably seal the distal opening, the opening mechanism including a cover coupled to the receptacle body, and a lever configured to transition the opening mechanism between a closed configuration and an opened configuration.

2. The non-invasive urinary output measuring system according to claim 1,
wherein the receptacle body includes a collection port having a lumen therethrough, extending from the collection cavity to an external surface of the receptacle body.

3. The non-invasive urinary output measuring system according to claim 1,
wherein the receptacle body includes a seat.

4. The non-invasive urinary output measuring system according to claim 3, wherein the lever is coupled to the seat.

5. The non-invasive urinary output measuring system according to claim 4, wherein the opened configuration includes the lever in a distal position and the cover is detached from the distal opening.

6. The non-invasive urinary output measuring system according to claim 4, wherein the closed configuration includes the lever in a proximal position and the cover seals the distal opening.

7. The non-invasive urinary output measuring system according to claim 3, wherein the fluid sensor includes a laser fluid sensor or an ultrasound fluid sensor.

8. The non-invasive urinary output measuring system according to claim 7, wherein the fluid sensor is coupled to the seat or to the internal hydrophobic surface.

9. The non-invasive urinary output measuring system according to claim 3, wherein the one or more light indicators are coupled to the seat.

10. The non-invasive urinary output measuring system according to claim 3, wherein the console is coupled to the seat or the receptacle body.

11. The non-invasive urinary output measuring system according to claim 1,
wherein the opening mechanism includes an activation lock configured to secure the opening mechanism in the closed configuration.

12. The non-invasive urinary output measuring system according to claim 1, wherein the plurality of logic modules, when executed by the processor, are configured to perform operations including:
detecting a volume of fluid within the collection cavity;
measuring a volume of fluid within the collection cavity;
associating a volume measurement value with a time of day value in a volume measurement value-time of day value pairing;
illuminating the light indicator a first color;
activating an activation lock;
illuminating the light indicator a second color; and
transmitting the volume measurement value-time of day value pairing to a display or a computing device.

13. The non-invasive urinary output measuring system according to claim 12, wherein the light indicator is configured to illuminate the first color when the console is obtaining a volume measurement value.

14. The non-invasive urinary output measuring system according to claim 13, wherein the activation lock is activated while the light indicator illuminates the first color.

15. The non-invasive urinary output measuring system according to claim 12, wherein the light indicator is configured to illuminate the second color when the console has received the volume measurement value-time of day value pairing or the console has transmitted the volume measurement value-time of day value pairing to the display or the computing device.

16. The non-invasive urinary output measuring system according to claim 1,
wherein the internal hydrophobic surface includes a plurality of super hydrophobic structures thereon.

17. The non-invasive urinary output measuring system according to claim 1, wherein the opening mechanism is biased to the closed configuration.

18. The non-invasive urinary output measuring system according to claim 1, wherein the cover includes a hinge.

19. A method of measuring a volume of fluid from a user, comprising:
acquiring a volume of fluid within a collection cavity of a non-invasive urinary output monitoring system, the collection cavity having an internal hydrophobic surface;
illuminating a light indicator of a fluid sensor system, a first color;
detecting a volume measurement value using the fluid sensor system;
illuminating the light indicator a second color; and
disposing of the volume of fluid using an opening mechanism.

20. The method according to claim 19, wherein illuminating a light indicator of the fluid sensor system, a first color includes illuminating the light indicator in communication with a console a first color.

21. The method according to claim 20, wherein illuminating a light indicator of the fluid sensor system, a first color includes illuminating the light indicator coupled to a seat of a urinary output measuring device of the non-invasive output monitoring system, a first color.

22. The method according to claim 19, wherein detecting the volume measurement value using the fluid sensor system includes detecting the volume measurement value using a fluid sensor in communication with the console.

23. The method according to claim 22, wherein detecting the volume measurement value using the fluid sensor system includes detecting the volume measurement value using the fluid sensor wherein the fluid sensor is a laser fluid sensor or an ultrasound fluid sensor.

24. The method according to claim 23, wherein detecting the volume measurement value using the fluid sensor system includes detecting using the fluid sensor when the fluid sensor is coupled to a seat or to the internal hydrophobic surface.

25. The method according to claim 19, wherein disposing of the volume of fluid using an opening mechanism includes obtaining a sample of the volume of fluid through a lumen of a collection port extending from the collection cavity to an external surface of a receptacle body having the collection cavity therein.

26. The method according to claim 25, wherein disposing of the volume of fluid using the opening mechanism includes transitioning the opening mechanism from a closed configuration to an opened configuration to dispose of the volume of fluid.

27. The method according to claim 26, wherein transitioning the opening mechanism from the closed configuration to the opened configuration includes moving a lever from a proximal position in the closed configuration to a distal position in the opened configuration.

28. The method according to claim 27, wherein transitioning the opening mechanism from the closed configuration to the opened configuration includes a cover configured to detachably seal a distal opening of the collection cavity transitioning from a closed configuration sealing the volume of fluid in the collection cavity to an opened configuration allowing the volume of fluid to exit the collection cavity.
